(19) United States
(12) Patent Application Publication
(10) Pub. No.: US 2011/0040349 A1
(43) Pub. Date: Feb. 17, 2011

(54) Noninvasive electrical stimulation system for standing and walking by paraplegic patients

(76) Inventor: Daniel Graupe, Hightland Park, IL (US)

Correspondence Address: DANIEL GRAUPE, 496 HILLSIDE DRIVE, HIGHLAND PARK, IL, 60035, US

(21) Appl. No.: 12/462,976

(22) Filed: Aug. 12, 2009

(51) Int. Cl. A61N 1/36

(52) U.S. Cl. 607/49

(57) ABSTRACT

The present invention is concerned with functional electrical stimulation (FES) of paraplegics having spinal cord injuries (SCI), especially for the purpose of walking, where stimulation is applied to motor neurons below the level of the SCI. Specifically, the invention is concerned with FES in closed-loop where closed loop operation is provided by wireless feedback by EMG signals recorded via noninvasive surface EMG electrodes. No wire connections are required between the EMG electrodes and a signal processor (SP) for providing the feedback signal to the SP. Also, no wire feedback is required to send timing information from the stimulation signal generator to blocking circuits, in cases where such circuits are required to protect the wireless transmitters of the feedback information from being damaged by the stimulation pulses. Wireless operation is facilitated by miniature chips (receivers and transmitters), such as used in the Bluetooth technology. Hence, the paraplegic users are not burdened with any wires that are otherwise needed for closed-loop operation and with the need to connect them between the patient's back, legs, and a pocket-borne control box. Furthermore, closed loop operation frees the patients from the need to manually adjust stimulation levels with progression of muscle fatigue.

The present invention allows the achieving closed-loop FES without requiring the sharing the same electrode for both stimulation and EMG recording and which requires complex control and non-standard electrodes. The avoidance of electrode-sharing further allows using regular and widely available stimulation electrodes and regular surface EMG electrodes, such as described in Graupe and Kohn: “Functional Electrical Stimulation for Ambulation by Paraplegics”, 1994.

In certain realizations of the present invention, the blocking circuit discussed above requires no input from the stimulus signal generator, while such inputs are essential in any electrode-sharing design since pulse level is highest at the stimulation site. Hence, also no wireless receiver is required next to the EMG electrodes and no wireless transmitter is required next to the stimulus signal generator.

In certain other realizations, blocking circuits are not required at all.

### FIELD OF THE DISCLOSURE

The present disclosure relates generally to functional electrical stimulation (FES), and more specifically to a method and an apparatus for managing spinal-cord-injured paraplegics by closed-loop functional electrical stimulation for standing and for walking

### BACKGROUND

Spinal cord injuries (SCI) and related trauma to the spinal cord, when resulting in the total or significant severance of the spinal cord, results in paralysis, in terms of loss of motor function and of sensation below the level of the lesion. When the lesion is an upper-motor-neuron (UMN) lesion, then the motor neurons below the lesion respond to electrical stimulation. Such lesions in the thoracic level (T1-T12) of the spinal cord, result in loss of motor function and sensation in the lower extremities, such that the patients lose their ability to stand and to walk. The motor neurons at below the lesion are then intact but can no more receive neurological commands from the brain, since the commands cannot reach beyond the lesion in the spinal cord. Since the below-lesion motor neurons in such upper-motor-neuron lesions are intact, they can respond to functional electrical stimulation (FES) when properly generated and applied (See: D Graupe and K H Kohn: “Functional Electrical Stimulation for Ambulation by Paraplegics”, Krieger Publ. Co., 1994 and D. Graupe, H Cerrel-Bazo, H Kern and U Carraro: “Walking performance, medical outcomes and patient training in FES of innervated muscles for ambulation by thoracic level complete paraplegics”, Neurological Research, 30, 2, 123-130, 2008). Also, see FIG. 1. The FES system discussed in these two reference was invented and developed by D Graupe and received FDA approval in 1994 (Approval No. P900038. See: http:/www.fda.gov/cdrh/pma94 htnl, April 1994),

Since the surface EMG (electromyographic) signal is a spatial integration of action potential (AP) in motor-neurons of the muscles at the recording site on the skin surface over a given muscle, no EMG exists can be recorded at that site. However, FES applied at a given muscle site below a UMN spinal-cord lesion, triggers APs in the motor neurons to result in contraction (innervation) of these muscles. Therefore an EMG signal does appear at that site during stimulation.

In paralysis as above, not only motor function is lost below the CSI lesion, but so is sensation. Hence, paraplegics who stand and walk with FES, require a walker (as in FIG. 2) for balance. In rare, low level lesions (T11-T12, some patient may walk with canes.

In D Graupe (U.S. Pat. No. 5,070,873, issued Dec. 10, 1991 an FES system is described that uses EMG feedback where the same electrode that stimulates the group of motor neurons at a given muscle also records the EMG that is generated (in response to the stimulation) at that same muscle, as discussed in [0003].

Electrode sharing as in [0005] is facilitated by a blocking circuit (BLK) which is operated to switch between connecting the electrode on that given muscle between a stimulation mode (SM) where it served to connect the stimulation signal generator (SG) to that electrode and a recording mode (RM) where it sends the EMG signal to a signal processing (SB) sub-unit that controls the SG.

The BLK circuit not only directs the traffic to/from the given electrode (to allow it to serve in a dual purpose manner) but also avoids the stimulation pulse (which is much stronger that the recorded EMG signal) from reaching and damaging the SP sub-unit. Hence, it receives input from the SG so that it can switch the RM mode a very short time (say a few milliseconds) before the start of the stimulus pulse that is generated in the SG. Similarly, input from SG also reconnects the RM mode a very short time (say, a few milliseconds) after the end of the stimulus pulse.

It is noted that without the blocking [0006-0007] of the stimulation pulse from the SP, the system, as in Graupe (U.S. Pat. No. 5,070,873), cannot function, since the SP will be severely damaged by the strong stimulation pulse.

In the design of (Graupe: U.S. Pat. No. 5,070,873) an input from the SG to the BC is essential to blocking, since BC must know when the stimulus starts and when it stops.

The design in (Graupe: U.S. Pat. No. 5,070,873) is specifically concerned with designs where electrode sharing [0005] is employed (claims 2 and 26 and all Figures of U.S. Pat. No. 507,873), namely for cases where the same electrode serves for both stimulation and EMG recording and where connection to the stimulation control unit is by wire. Electrode sharing requires specially designed electrodes and the stimulation signal's high voltage level is beyond what a wireless transmitter can handle, especially if it is to be incorporated with any skin electrode glued to the patient's body.

### BRIEF SUMMARY OF THE INVENTION

The present invention is concerned with functional electrical stimulation (FES) of paraplegics having spinal cord injuries (SCI), especially for the purpose of independent walking, where stimulation is applied to motor neurons below the level of the SCI-lesion. The present improvement aims at providing FES in closed-loop, where all feedback is linked to the main FES system by wireless. Such automatic feedback serves to enhance patient independence, as it relieves the patient of manually adjusting stimulation levels to compensate for muscle fatigue. Furthermore, when all feedback links are wireless, then feedback does not involve additional wires between the patient's limbs, back and the chest-pocket-borne or belt-attached stimulation control unit. Also, when setting up the electrodes every morning or removing them in the evening, the patient need not connect a multitude of wires for the feedback links.

Specifically, the invention is concerned with closed-loop FES where feedback is provided by wireless from EMG signals recorded via noninvasive surface EMG electrodes. No wire connections are required between the EMG electrodes and a signal processor (SP) for providing the feedback signal to the SP. Also, no wire feedback is required to send timing information from the stimulation signal generator to blocking circuits, in cases where such circuits are required to protect the wireless transmitters of the feedback information from being damaged by the stimulation pulses. Wireless operation is facilitated by miniature chips (receivers and transmitters), such as used in the Bluetooth technology. Hence, the paraplegic users are not burdened with any wires that are otherwise needed for closed-loop operation and with the need to connect them between the patient's back, legs, and a pocket-borne control box. Furthermore, closed loop operation frees the patients from the need to manually adjust stimulation levels with progression of muscle fatigue.

An earlier design for feedback FES (D. Graupe: U.S. Pat. No. 5,070,873) is specifically concerned with employing electrode sharing [0005], [0010] (claims 2 and 26 and all Figures of U.S. Pat. No. 507,873), namely for cases where the same electrode serves for both stimulation and EMG recording and where connection to the stimulation control unit is by wire. Electrode sharing requires specially designed electrodes. Also, the high voltage level of the stimulus pulse is beyond what a wireless transmitter can handle, especially if it is to be incorporated with any skin electrode glued to the patient's body. The present invention allows the achieving closed-loop FES without requiring the sharing the same electrode for both stimulation and EMG recording and which requires complex control and non-standard electrodes. The avoidance of electrode-sharing further allows using regular and widely available stimulation electrodes and regular surface EMG electrodes, such as described in Graupe and Kohn: “Functional Electrical Stimulation for Ambulation by Paraplegics”, 1994.

Also, adequate placement of the EMG electrodes will considerably reduce the effect of the stimulus pulse at the recording site, noting that this effect is maximal at the stimulation site, namely, where shared electrodes would have been placed. Hence, also no wireless receiver is required next to the EMG electrodes and no wireless transmitter is required next to the stimulus signal generator in these realizations. Furthermore, in certain other realizations, blocking circuits are therefore not required at all.

### BRIEF DESCRIPTION OF THE DRAWINGS

FIG. 1: Paraplegic Patient Walking with FES System

FIG. 2: FES System Schematic

FIG. 3: FES Stimulation Electrodes and EMG Recording electrodes on Quadriceps Muscles of Paraplegic Patient (electrodes are not shown)

FIG. 4: Block Diagram of the Closed-Loop FES System

### DETAILED DESCRIPTION

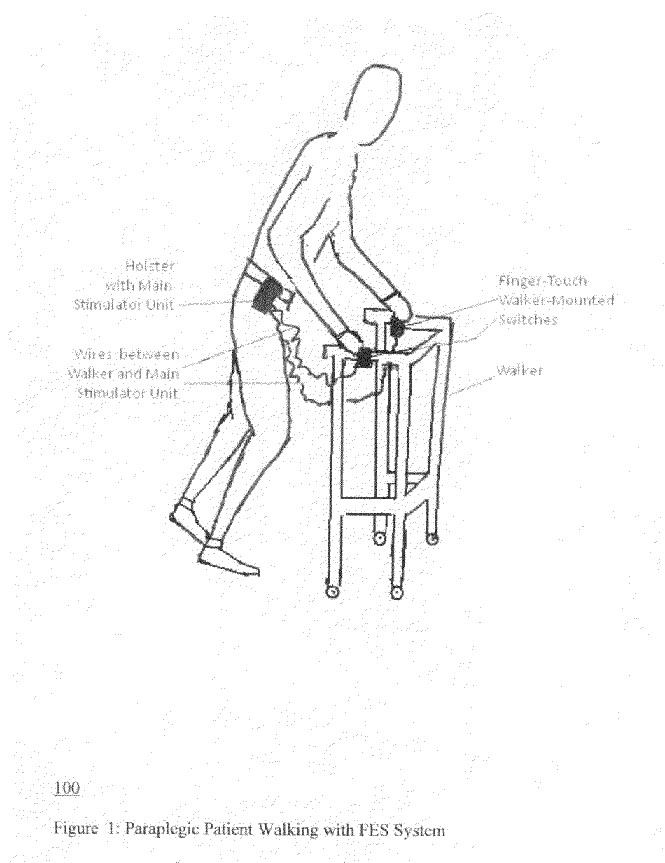

This invention is of an improved noninvasive functional electrical stimulation (FES) method and device to provide closed-loop control of the stimulation in order to enhance patient independence and to simplify the operation of the system by a paralyzed patient during standing and walking with FES, as in FIG. 1 [00015]

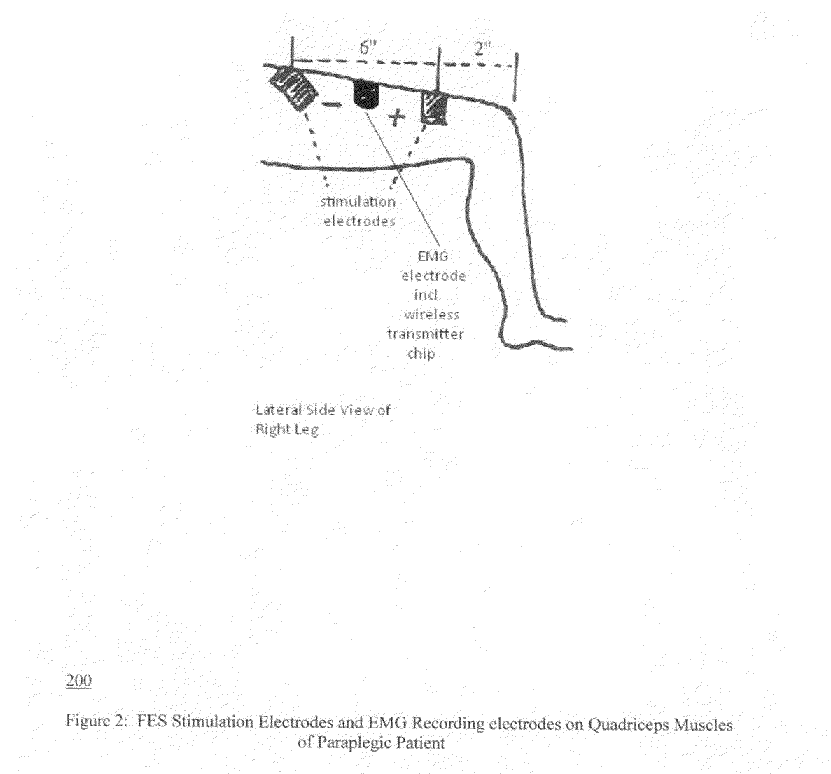

The closed loop control is established via placing noninvasive EMG (electromyographic) electrodes on the surface of the skin above the muscles which undergo FES, as in the example shown in FIG. 2 [0016]

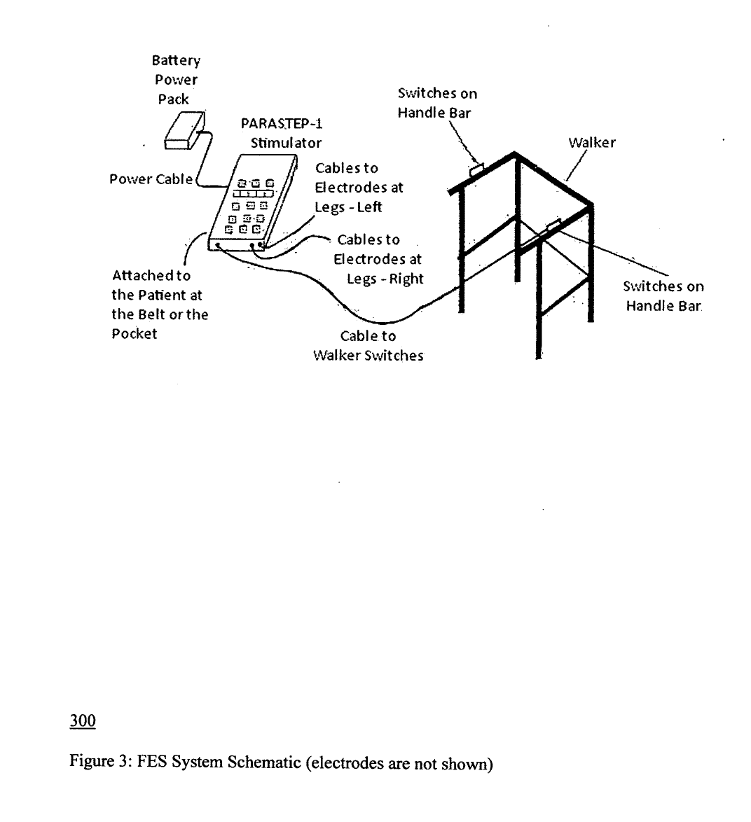

A preferred realization of the FES system but without the EMG electrodes assembly (which includes a wireless transmitter) and without the stimulation electrodes is as in FIG. 3 [0017].

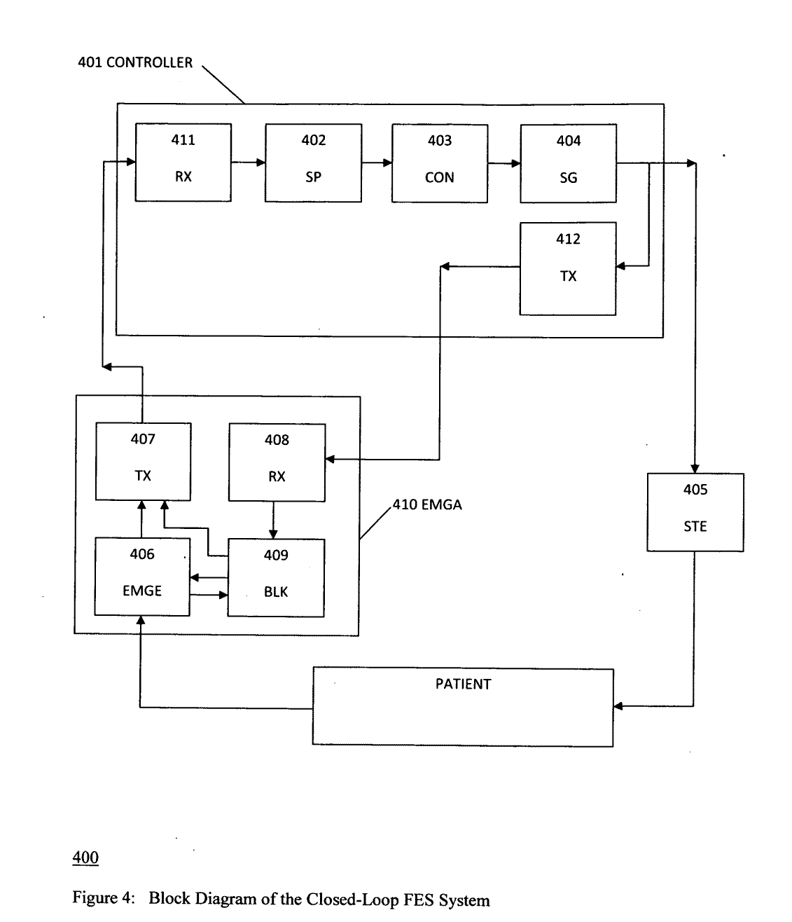

FIG. 4 [0018] describes the closed-loop structure and operation of the system in further detail, as follows:

The stimulation controller (C) as in block 401 is the brains of the FES system. It incorporates a signal processing (SP) sub-unit the feedback signals 402 which feeds its processed information to a stimulation controller CON sub-unit 403, which, in turn, controls the signal generation SG 404 sub unit, where the stimulation signal are generated and distributed to the various noninvasive (surface) stimulation electrodes STE 405 which apply trains of stimuli transcutaneously at the various sites where muscle contractions are required for walking and for trunk stability (See: D Graupe and K H Kohn: “Functional Electrical Stimulation for Ambulation by Paraplegics”, Krieger Publ. Co., 1994). Muscle contractions result from the action potentials that are being triggered repeatedly (at a rate of 20 to 30 pulses per second) by these stimuli in the appropriate groups of motor neurons (see: [0002] and D. Graupe, H Cerrel-Bazo, H Kern and U Carraro: “Walking performance, medical outcomes and patient training in FES of innervated muscles for ambulation by thoracic level complete paraplegics”, Neurological Research, 30, 2, 123-130, 2008).

The muscle contraction results in EMG activity (see [0003] above) that exists not just during the FES stimulus pulse duration (of approximately 100 microsecond) but also over the interval between adjacent pulses (stimuli). See: D Graupe and K H Kohn: “Functional Electrical Stimulation for Ambulation by Paraplegics”, Krieger Publ. Co., 1994. These are recorded at the EMG electrodes EMGE 406 in FIG. 4 (also see FIG. 2). These signals are transmitted by (miniature) wireless transmitter TX 407 chips, such as using Bluetooth technology, which are physically incorporated in the EMG electrode assembly EMGA 410 of FIG. 4 to wireless receiver RX 411 that may be located in the Controller unit 401.

The loop is closed by the wireless receiver RX 411 of FIG. 4 that is incorporated in the Stimulation Control unit 401 of FIG. 4. RX 411 then passes the received signals to SP 402 for processing, as in [0022], etc.

The action potential of the stimulated motor neurons and the resulting surface EMG signal as recorded at stimulated sites lasts for a large portion if not all the interval (of the order of 4 to 5 milliseconds) between two successive stimuli, while the stimulation pulse (namely, the stimulus) lasts only approximately 0.1 milliseconds (see [006], [007]). However, the EMG signal is still usually stronger than the EMG signal, even if separate electrodes are used for stimulation and for EMG-recording, and these are placed at an appropriate distance from one another. Hence, in some realizations damage can be caused to the EMG transmitter TX 407 by the effect of the stronger though short stimulus. Hence, in several realizations a blocking circuit BLK 409 of FIG. 4 is incorporated in the EMG assembly EMGA 410. BLK 409 serves to guarantee that the EMG signal does not include voltage values above a threshold that may damage the wireless transmitter TX 407.

Blocking in circuit BLK 409 of FIG. 4 can be done via a voltage limiter.

Alternatively blocking can be done by incorporating a wireless receiver RX 408 of FIG. 4, that receives information on the start and stop of the stimulus from stimulus generator SG 404, via wireless transmitter TX 412.

TX 412 will usually be housed in the controller unit 401 of FIG. 4, where also SG 403 is housed, while EMGE 406, TX 407, RX 408 and BLK 409 will usually be housed in the EMG assembly EMGA 410, where TX 407, RX 408 and BLK may be all in a single microchip.

In other realizations, no information from SG 404 of FIG. 4, and hence, no link to SG 404 may be required for blocking in BLK 409, Hence, RX 408, TX 412 and the link between them are not required, whereas blocking in BLK 409 will be accomplished by voltage limiting

In still other realization, no blocking for protecting transmitter 407 of FIG. 4 will be needed at all. Hence, EMGE may be connected directly to TX 407. Alternatively BLK 407 will serve solely to match impedances and/or voltage levels between EMGE 406 and TX 407.

What is claimed is:

1. An FES stimulation device comprising of
a simulation control unit in which signal processing and stimulation control are performed and coordinated and which incorporates
a stimulation signal generator,
several stimulation electrodes,
a walker unit where manual control switches are installed,
one or more pairs of noninvasive surface EMG recording electrodes
and
wireless links to interconnect the various EMG electrodes and the walker unit with the stimulation control unit
and where noninvasive surface EMG electrodes are placed on same muscles that are being stimulated to record the EMG in these muscles arises in response to the FES stimulation.

2. A device as in claim 1, and where said EMG electrodes are housed in an EMG assembly (EMGA) at each EMG recording site,
and where the EMG assembly incorporates
a wireless transmitter circuit that transmits the recorded EMG signal from the EMG electrodes to a wireless receiver circuit that is incorporated with the signal processing sub-unit of the stimulation control unit,
and where said wireless receiver circuit receives the said transmitted EMG signal and serves to pass the information of said EMG signal to a signal processing sub-unit that may be located in the stimulation control unit.

3. A device as in claim 2, and where
said EMG electrodes send their signal to the wireless transmitter via a blocking circuit
and where the blocking circuit serves to block high voltages portions of the EMG signal that are due to effects of the stimulation pulse from damaging the wireless transmitter that transmits the EMG signal to the signal processor of the stimulation control unit

4. A device as in claim 3 and where
the blocking circuit includes a voltage limiter

5. A device as in claim 3 and where
the blocking circuit receives timing information through a wireless receiver from the stimulation signal generator on the timing of the beginning and of the ending of each stimulation pulse
and where the said timing information passes from a timing circuit that is part of the stimulation signal generator via a wireless transmitter
and where said wireless receiver is a miniature receiver that is incorporated in the EMG assembly of each EMG recording site
and where said wireless transmitter is housed with the stimulation control unit.

6. A device as in claim 2, and where
stimulation control unit processes the EMG signal that are received from the EMG electrodes

7. A device as in claim 1, and where
signal processing is via extracting EMG parameters

8. A device as in claim 7, and where
extraction is via a wavelet transform

9. A device as in claim 7, and where
extraction is via Least Squares identification, such as in D. Graupe: “Time Series Analysis, Identification and Adaptive Filtering”, Second Edition, Krieger Publ. Co., 1989.

10. A device as in claim 6, and where
a neural network such as in D. Graupe: “Artificial Neural Networks”, Second edition, World Scientific Publishers, 2007, serves to relate the EMG signal's parameters to level of muscle fatigue at stimulated site.

11. A device as in claim 1, and where
level of muscle fatigue at stimulated site, as derived from processed EMG signals, is used to automatically adjust stimulation levels at corresponding stimulation site and at other such sites up to a maximal predetermined level, to counter effects of such muscle fatigue

12. A device as in claim 6, and where
level of muscle fatigue at stimulated site, as derived from processed EMG signals, is used to automatically adjust stimulation levels at corresponding stimulation site and at other related sites up to a maximal predetermined stimulus-level, to counter effects of such muscle fatigue

13. A method for FES stimulation where
noninvasive surface EMG electrodes are placed on same muscles that are being stimulated to record the EMG as exists in these muscles in response to the FES stimulation.
and where
a wireless transmitter circuit transmits the recorded EMG signal from the EMG electrodes to a stimulator controller
and where
a wireless receiver receives the transmitted EMG signal and may be incorporated with the simulation control sub-system.

14. A method as in claim 13, and where
the stimulation controller processes the EMG signal that are received from the EMG electrodes

15. A method as in claim 14, and where
level of muscle fatigue at stimulated site, as derived from processed EMG signals, is used to automatically adjust stimulation levels at corresponding stimulation site and at other related sites up to a maximal predetermined stimulus level, to counter effects of such muscle fatigue.

16. A method as in claim 13, and where
said EMG electrodes send their signal to the wireless transmitter via a blocking circuit
and where the blocking circuit serves to block high voltages portions of the EMG signal that are due to effects of the stimulation pulse from damaging the wireless transmitter that transmits the EMG signal to the signal processor of the stimulation control sub-system

17. A method as in claim 16, and where
the blocking circuit receives timing information through a wireless receiver from the stimulation signal generator on the timing of the beginning and of the ending of each stimulation pulse
and where the said timing information passes from a timing circuit that is part of the stimulation signal generator via a wireless transmitter
and where said wireless receiver is a miniature receiver that is incorporated in the EMG assembly of each EMG recording site.
